(19) United States
(12) Patent Application Publication
(10) Pub. No.: US 2011/0300566 A1
(43) Pub. Date: Dec. 8, 2011

(54) METHODS FOR SCREENING FOR HISTONE DEACETYLASE ACTIVITY AND FOR IDENTIFYING HISTONE DEACETYLASE INHIBITORS

(76) Inventor: Dewey G. McCafferty, Plymouth Meeting, PA (US)

(21) Appl. No.: 13/149,293

(22) Filed: May 31, 2011

Related U.S. Application Data
(63) Continuation of application No. 10/444,117, filed on May 23, 2003, now abandoned.
(60) Provisional application No. 60/383,308, filed on May 23, 2002.

(51) Int. Cl. C12Q 1/34

(52) U.S. Cl. 435/18

(57) ABSTRACT

The present invention relates to novel methods for screening for histone deacetylase enzyme activity in a test sample. The present invention further relates to novel methods for screening potential inhibitors of histone deacetylase enzymes.

This application claims the benefit of U.S. Provisional Patent Application No. 60/383,308, filed May 23, 2002, the contents of which is hereby incorporated by reference in its entirety.

This work was supported in part by grant number GM65539 from the National Institutes of Health. The United States government may have rights in this invention by virtue of this support.

### BACKGROUND OF THE INVENTION

1. Field of Invention

The present invention relates to novel methods for screening for histone deacetylase enzyme activity in a test sample. The present invention further relates to novel methods for screening potential inhibitors of histone deacetylase enzymes.

2. Background

Histones and Gene Expression. In eukaryotic cells, nuclear DNA associates with histones to form a compact complex called chromatin. The histones constitute a family of basic proteins which are generally highly conserved across eukaryotic species. The core histones, termed H2A, H₂B, H3, and H4, associate to form a protein core. Histones are the protein portion of a protein-DNA complex termed the nucleosome. DNA winds around this protein core, with the basic amino acids of the histones interacting with the negatively charged phosphate groups of the DNA. Nucleosomes structurally organize chromosomal DNA to form chromatin. The repeating structural motif of chromatin is the nucleosome particle, which is comprised of approximately 146 base pairs of DNA wrapped around a histone core.

Histones are subject to post-translational acetylation of the ε-amino groups of N-terminal lysine residues, a reaction that is catalyzed by histone acetyl transferase. The degree of interaction between histones and DNA varies between regions undergoing transcription and regions not being transcribed in normal cells. The histones in chromatin regions under active transcription are often post-translationally modified with acetyl groups covalently attached to specific lysine residues, which has been both positively and negatively correlated with gene activity. Discovered nearly 40 years ago, it has only recently become clear that acetylation of the ε-amino group of specific lysine residues within the positively charged N-terminal tail of core histones H2A, H₂B, H3, and H4 results in localized chromatin relaxation and a change in both histone-DNA and histone-nonhistone protein interaction.

Histone deacetylase (hereinafter “HDAC”) and histone acetyltransferase together control the net level of acetylation of histones and maintain the delicate dynamic equilibrium in the acetylation level of nucleosomal histones. In general, acetylation activity is correlated with transcriptional activation, whereas deacetylation activity is accompanied by transcriptional repression. Acetylation neutralizes the positive charge of the lysine side chain, and is thought to impact chromatin structure. Access of transcription factors to chromatin templates is enhanced by histone hyperacetylation. An enrichment in underacetylated histone H4 has been found in transcriptionally silent regions of the genome.

Histone acetylation is a reversible modification, with deacetylation being catalyzed by members of the histone deacetylase family of enzymes. Histone deacetylases are responsible for de-acetylation and may be localized to DNA targeted for repression by other proteins that associate with HDAC and specifically bind regulatory elements in genes. HDACs are divided into two classes, Class I represented by yeast Rpd3-like proteins, and Class II represented by yeast Hda1-like proteins. Human HDAC1, HDAC2, HDAC3, and HDAC8 proteins are members of the Class I group of HDACs. Human HDAC4, HDAC5, HDAC6, and HDAC7 are members of the Class II group of HDACs.

Inhibition of the action of histone deacetylase results in the accumulation of hyperacetylated histones, which in turn is implicated in a variety of cellular responses, including altered gene expression, cell differentiation, and cell-cycle arrest. Hyperacetylated histones are thought to adopt a chromatin structure that allows other proteins to activate DNA transcription. Inactive genes are associated with hypoacetylated histones, and removal of the acetyl groups from histones in normally active chromatin will repress transcription in that region. The reversible acetylation of histones is crucial for the transcriptional regulation of gene expression in eukaryotic cells.

HDAC activity is inhibited by trichostatin A (TSA), a natural product isolated from Streptomyces hygroscopicus, and by a synthetic compound, suberoylanilide hydroxamic acid (SAHA). TSA arrests development of rat fibroblasts at the G₁ and G₂ phases of the cell cycle, implicating HDAC in cell cycle regulation. TSA and SAHA inhibit cell growth, induce terminal differentiation, and prevent the formation of tumors in mice. These findings suggest that inhibition of HDAC activity represents a novel approach for intervening in cell cycle regulation and that HDAC inhibitors have great therapeutic potential in the treatment of cell proliferative diseases or conditions. To date, only a few inhibitors of histone deacetylase are known in the art. There is thus a great need to identify additional HDAC inhibitors and to identify the structural features required for potent HDAC inhibitory activity.

Coumarin Derivatives. 2-H-1-benzopyran-2-one, commonly known as Coumarin, is the parent organic compound of a class of naturally occurring phytochemicals found in many plant species. An oxygen heterocycle, it is best known for its fragrance, described as a vanilla-like odor or the aroma of freshly mowed hay. Identified in the 1820s, coumarin has been synthesized in the laboratory since 1868 and used to make perfumes and flavorings. Chemically, coumarin can occur either free or combined with glucose, to produce a coumarin glycoside derivative. Medically, other coumarin glycoside derivatives have been shown to have blood-thinning, anti-fungicidal, and anti-tumor activities. Dicumarol, a coumarin glycoside better known as warfarin, is a commonly used oral anticoagulant. It undergoes very extensive metabolism along two major pathways, 7-hydroxylation and ring-opening to ortho-hydroxyphenylacetaldehyde. The relative extent of these two major pathways is highly variable between species. Ring-opening predominates in rodents, while 7-hydroxylation is particularly evident in humans.

Coumarin is the basic structure of numerous naturally occurring compounds with important and diverse physiological activities. More than a thousand coumarin derivatives have been described, varying from simple coumarins containing alkyl and hydroxyl side chains to complex coumarins with benzoyl, furanoyl, pyranoyl, or alkylphosphorothionyl substituents. The structures of coumarin and some of its most common pharmaceutical derivatives are shown below.

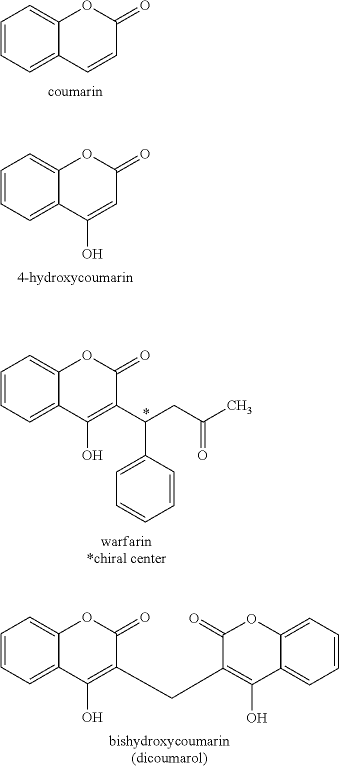

Additionally, a number of coumarin derivatives have fluorescence properties and are used as dye molecules; an example are the 7-aminocoumarins, which exhibit characteristic, molecule-specific peak absorption and fluorescence emission spectra. For example, a common laser dye, 7-diethylamino-4-methylcoumarin or Coumarin 1, has a peak absorption wavelength of about 373 nm and a peak emission wavelength of about 460 nm. Other common exemplary coumarin derivatives with known fluorescence properties include Coumarin 6, 30, 35, 102, 120, 138, 151, 152, and 153. The fluorescence properties of a large number of coumarin derivatives, having fluorescence emission spectra over a wide range, are known of to those of ordinary skill in the art.

Prior Art HDAC Assays. The characterization of HDAC activity has been problematic for many reasons. First, it is difficult to obtain sufficient quantities of pure enzyme that retain catalytic activity. Histone deacetylases are zinc-dependent enzymes, and many of the methods established for purifying recombinant proteins with an engineered affinity tag require the use of chelators that may strip the enzyme of its metal. Second, all previously established in vitro activity assays are discontinuous or require the use of radioactivity. The acetate extraction method relies on the use of a [3H]-acetylated histone peptide substrate corresponding to the sequence of a histone tail. The HDAC enzyme is incubated with the substrate, and the reaction quenched with hydrochloric and acetic acid. Next the released [3H] acetic acid is extracted with ethyl acetate and quantified by scintillation counting. It has been shown that the solubility of acetic acid in ethyl acetate is limiting, causing an underestimate of 3H acetate removed.

The discovery of novel HDAC inhibitors as new drugs for transcription therapy and cancer chemoprevention is currently obstructed by the lack of suitable assay systems. One widely distributed assay of HDAC activity depends on the incubation of the enzyme with acetate-radiolabeled histones or peptide substrates, followed by extraction with organic solvents such as ethyl acetate and then the quantification of the released radiolabeled acetic acid by liquid scintillation count. 3H-histones are obtained by a laborious procedure that relies on the sacrifice of animals. The degree of acetylation of prelabeled core histones changes within different preparations and it is therefore difficult to standardize the substrate properties.

Similarly, labeled oligopeptides are synthesized by solid-phase technology, and postlabeling HPLC purification is required. Although classical radioactive assays have been successfully used to measure HDAC activities from various sources, the need to separate product from substrate limits assay throughput. In addition, the use of scintillation cocktails makes these assays costly in terms of time, labor, and radioactive waste. Hence, assays of this type are not readily amenable to automation and high-throughput screening.

A principally nonisotopic method for the determination of HDAC activity relied on immunoblotting of hyperacetylated histones. However, this approach has the drawback that rather than measuring enzyme activity in the presence or absence of inhibitor, these immunoblotting procedures more resembled functional tools that, are not suited for assay throughput. Another nonisotopic assay for HDAC activity used (N-(4-methyl-7-coumarinyl)-(tert-butyloxy-carbonyl)-acetyllysinamide) as a substrate. Unfortunately, formation of the deacetylated product is monitored by HPLC and fluorescence detection after extraction with ethyl acetate. As a result, these prior art assays are not well suited for high-throughput screening.

In addition, the substrates used in the prior art do not well resemble acetylated lysine residues in the original context of histones. Recent improvements of this assay include: (1) fluorescence-labeled octapeptide substrates that bear some closer resemblance to the native substrate; and the introduction of an internal standard for the quantification of fluorescence substrate by HPLC. Although the prior art assays have been used with limited success, including for the study of time- and site-dependent deacetylation, they still remain not readily amenable to automation and high-throughput screening due to the required separation steps.

Other assays for histone deacetylase activity can be used as a preliminary screen to select candidates for other differentiation agents. For example, U.S. Pat. No. 5,922,837 discloses an assay using tritiated desmethoxyapicidin and a parasite or chick liver 5100 solution as a source of deacetylase activity. The candidate compound is added to the reaction mixture, and tritium release is measured using a filter method.

Nare et al., Anal. Biochem. 267:390 (1999) have developed a scintillation proximity assay using a peptide from histone H4, with lysine ε-amino groups acetylated with tritium, and bound to an SPA bead that scintillates proportionately to the amount of proximal tritium. Histone deacetylase activity, obtained from extracts of HeLa cell nuclei, releases the labeled acetyl groups and decreases scintillation, and the presence of a deacetylase inhibitor maintains scintillation.

Hoffman et al., Nucl. Acids Res. 27:2057 (1999) describes a non-isotopic assay for histone deacetylase activity. A fluorescent substrate has been developed that is an aminocoumarine derivative of Ω-acetylated lysine. This permits quantitation of substrate in the nanomolar concentration range, which allows for high throughput screening of histone deacetylase inhibitors.

Manfred Jung and colleagues have developed a number of HDAC assays that do not rely on radioactivity. The first non-isotopic assay utilizes a fluorescent derivative of ε-acetyl lysine, (N-(4-methyl-7-coumarinyl)-α-(tert-butyloxy-carbonyl)-W-acetyllysinamide), which can be quantified using a reverse-phase HPLC system with a fluorescence detector. An internal standard was later employed to improve the accuracy of the assay. Fluorescein-labeled octapeptides were designed to more closely resemble the native HDAC substrate and constructed for use in another HPLC and fluorescence detection based assay. Their studies revealed a preference for the C-terminal acetyl-lysine position, but this finding may be due to steric hindrance associated with the N-terminal fluorescein group.

Recently, a commercially developed kit, which comprises a proprietary acetylated lysine side chain substrate, was introduced by the Plymouth Meeting, Pennsylvania company BIOMOL Research Laboratories, Inc., for high-throughput screening of HDAC activity in a 96-well plate format. In this assay, HDAC enzyme is incubated with the substrate, the reaction is quenched with a proprietary developer solution, and the resulting fluorescent signal is quantified using a fluorescence microtiter plate reader.

U.S. Pat. No. 6,428,983 issued Aug. 6, 2002 to Dulski, et al. discloses a method for identifying compounds that are antiprotozoal agents by determining whether a test compound or natural product extract inhibits the action of protozoal histone deacetylase, said method comprising: (a) contacting a protozoal histone deacetylase, or an extract containing protozoal histone deacetylase with a known amount of a labeled compound that interacts with a protozoal histone deacetylase in the presence or absence of a known dilution of a test compound or a natural product extract; and (b) quantitating the percent inhibition of interaction of said labeled compound by the difference in the enzyme activities in the presence and absence of the inhibitor test compound or natural product extract. Dulski, et al. disclose that substrates for histone deacetylase may be acetylated histones, or a labeled acetylated peptide fragment derived therefrom such as AcGly-Ala-Lys(ε-Ac)-Arg-His-Arg-Lys(ε-AC)-ValNH₂, or other synthetic or naturally occurring substrates. Examples of known compounds that bind to histone deacetylase are known inhibitors such as α-butyrate, trichostatin, trapoxin A, and other inhibitors.

Wegener D, Wirsching F, Riester D, Schwienhorst A, A fluorogenic histone deacetylase assay well suited for high-throughput activity screening, Chem. Biol. January; 10(1):61-8 (2003), which was published after the priority date of this application, discloses a fluorogenic assay for HDAC activity for expediting studies of HDAC in transcriptional regulation and in vitro screening for drug discovery. In that work, fluorogenic substrates of HDACs were synthesized with an epsilon-acetylated lysyl moiety and an adjacent MCA moiety at the C terminus of the peptide chain. Upon deacetylation of the acetylated lysyl moiety, molecules became substrates for trypsin, which released highly fluorescent AMC molecules in a subsequent step of the assay. The fluorescence increased in direct proportion to the amount of deacetylated substrate molecules, i.e., HDAC activity. The primary drawback to fluorescent assays is that, although they are suitable for high-throughput screening and they alleviate the need for radioactive isotopes, they are all still discontinuous.

Thus, histone deacetylases are important enzymes for the transcriptional regulation of gene expression in eukaryotic cells. Recent findings suggest that HDACs could be key targets for chemotherapeutic intervention in malignant diseases. Compounds and compositions identified by the inventive methods are expected to be inhibitors of histone deacetylase, and are expected to inhibit cell proliferation in a contacted cell by growth retardation, growth arrest, programmed cell death, or necrotic cell death.

A convenient, sensitive, high-throughput assay for HDAC activity would therefore expedite studies of HDAC in transcriptional regulation and greatly improve in vitro screening for drug discovery. In response to this need, the inventive subject matter provides novel continuous assays for HDAC activity and provides for improved methods for selection of HDAC inhibitors. In particular, the inventive subject matter provides continuous, non-isotopic, spectrophotomeric—both fluorescent and chromogenic—assays for HDAC activity, and will greatly streamline the selection process for HDAC inhibitors, both generally and targeted to specific histone deacetylase enzymes.

### SUMMARY OF THE INVENTION

The present invention relates to a method for screening for histone deacetylase enzyme activity in a test sample, comprising the steps of:

i) contacting said test sample with a liquid screening solution comprising a compound of Formula I:

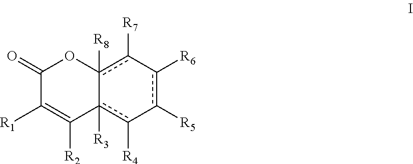

wherein:

R₁, R₂, R₃, R₄, R₅, R₆, R₇, and R₈ are independently selected from the group consisting of:

hydrogen,

hydroxyl,

carbonyl,

acetyl,

Ar,

straight or branched chain C₁-C₉ alkyl,

straight or branched chain C₁-C₉ alkyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

straight or branched chain C₂-C₉ alkenyl or alkynyl, and

straight or branched chain C₂-C₉ alkenyl or alkynyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

provided that at least one of R₁, R₂, R₃, R₄, R₅, R₆, R₇, and R₈ must be —O—C(O)—R₉, wherein R₉ is straight or branched chain C₁-C₃ alkyl; and

Ar is a mono-, bi- or tricyclic, carbo- or heterocyclic ring, wherein the ring is either unsubstituted or substituted in one or more position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, or amino; wherein the individual ring sizes are 5-6 members; and wherein the heterocyclic ring contains 1-6 heteroatom(s) selected from the group consisting of O, N, and S; and

ii) assaying for fluorescence produced by a product of histone deacetylase enzyme activity in said liquid screening solution.

The present invention further relates to a method for screening for inhibitors of histone deacetylase enzyme activity, comprising the steps of:

i) contacting an inhibitor candidate compound or composition with a liquid screening solution comprising a histone deacetylase enzyme and a compound of Formula I:

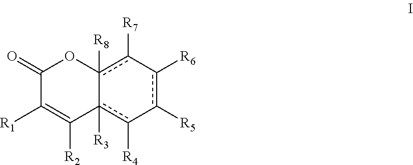

wherein:

R₁, R₂, R₃, R₄, R₅, R₆, R₇, and R₈ are independently selected from the group consisting of:

hydrogen,

hydroxyl,

carbonyl,

acetyl,

Ar,

straight or branched chain C₁-C₉ alkyl,

straight or branched chain C₁-C₉ alkyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

straight or branched chain C₂-C₉ alkenyl or alkynyl, and

straight or branched chain C₂-C₉ alkenyl or alkynyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

provided that at least one of R₁, R₂, R₃, R₄, R₅, R₆, R₇, and R₈ must be —O—C(O)—R₉, wherein R₉ is straight or branched chain C₁-C₃ alkyl; and

Ar is a mono-, bi- or tricyclic, carbo- or heterocyclic ring, wherein the ring is either unsubstituted or substituted in one or more position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, or amino; wherein the individual ring sizes are 5-6 members; and wherein the heterocyclic ring contains 1-6 heteroatom(s) selected from the group consisting of O, N, and S; and

ii) assaying for fluorescence produced by a product of histone deacetylase enzyme activity in said reaction solution.

### BRIEF DESCRIPTION OF THE DRAWINGS

FIG. 1 is a drawing which depicts the inventive process of monitoring the hydrolysis of 7AC to 7-hydroxycoumarin.

FIG. 2A is a drawing which depicts a MBP-Hos3 plasmid map.

FIG. 2B is a drawing which depicts the junction between Hos3 and MBP showing the TEV protease cleavage site in a MBP-Hos3 plasmid map.

FIG. 2C is a photograph which depicts an affinity purification gel of MBP-Hos3.

FIG. 3 is a graph which depicts a MALDI-TOF timecourse mass spectrograph of the deacetylation activity (−42 Da) of Hos3 enzyme on histone H4(1-24).

FIG. 4 is a graph which depicts IC₅₀ values of histone H4 (1-24) acetylated peptide, Trichostatin A, and sodium butyrate.

FIG. 5A is a graph which depicts Trichostatin A IC₅₀ values plotted as a function of substrate 7AC concentration.

FIG. 5B is a graph which depicts the Henderson plot of Kᵢ for Trichostatin A.

### DETAILED DESCRIPTION OF THE INVENTION

### Definitions

“Histone deacetylase” and “HDAC” refer to any one of a family of enzymes that remove acetyl groups from the ε-amino groups of lysine residues at the -terminus of a histone. Unless otherwise indicated, the term “histone” is meant to refer to any histone protein, including H1, H2A, H₂B, H3, H4, and H5, from any species. Preferred histone deacetylases include Class I and Class II HDAC enzymes.

“Histone deacetylase inhibitor” or “inhibitor of histone deacetylase” refers to a compound which is capable of interacting with a histone deacetylase and inhibiting its enzymatic activity. Inhibiting histone deacetylase enzymatic activity means reducing the ability of a histone deacetylase to remove an acetyl group from a histone.

“Test sample” refers to a sample which contains a test compound or natural product extract to be tested for histone deacetylase enzyme activity.

“Alkenyl” means a branched or unbranched unsaturated hydrocarbon chain comprising a designated number of carbon atoms. For example, C₂-C₆ straight or branched alkenyl hydrocarbon chain contains 2 to 6 carbon atoms having at least one double bond, and includes but is not limited to substituents such as ethenyl, propenyl, iso-propenyl, butenyl, iso-butenyl, tert-butenyl, -pentenyl, -hexenyl, and the like. It is also contemplated as within the scope of the present invention that “alkenyl” may also refer to an unsaturated hydrocarbon chain wherein any of the carbon atoms of said alkenyl are optionally replaced with O, NH, S, or SO₂. For example, carbon 2 of 4-pentene can be replaced with 0 to form (2-propene)oxymethyl.

“Alkoxy” refers to the group —OR wherein R is alkyl as herein defined. Preferably, R is a branched or unbranched saturated hydrocarbon chain containing 1 to 6 carbon atoms.

“Alkyl” means a branched or unbranched saturated hydrocarbon chain comprising a designated number of carbon atoms. For example, C₁-C₆ straight or branched alkyl hydrocarbon chain contains 1 to 6 carbon atoms, and includes but is not limited to substituents such as methyl, ethyl, propyl, iso-propyl, butyl, iso-butyl, tert-butyl, N-pentyl, N-hexyl, and the like. It is also contemplated as within the scope of the present invention that “alkyl” may also refer to a hydrocarbon chain wherein any of the carbon atoms of said alkyl are optionally replaced with O, NH, S, or SO₂. For example, carbon 2 of -pentyl can be replaced with O to form propyloxymethyl.

Throughout this application, “R” or “Rₙ”, where n is a number, is used to designate various substituents. These R groups are independently selected. Thus, for example, the fact that R₁ may be a branched alkyl in one context does not require that R₁ be the same branched alkyl, and does not prohibit that R₁ be, for example, a straight chain alkenyl in another context in the same molecule. It is intended that all “Rₙ” are selected independently of all other “Rₙ”, whether or not the term “independently selected” is used.

“Aryl” or “aromatic” refers to an aromatic carbocyclic or heterocyclic group having a single ring, for example a phenyl ring; multiple rings, for example biphenyl; or multiple condensed rings in which at least one ring is aromatic, for example naphthyl, 1,2,3,4-tetrahydronaphthyl, anthryl, or phenanthryl. The ring(s) of an aryl moiety can be unsubstituted or substituted with one or more substituents including, but not limited to, halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, or amino; a heterocyclic ring may contain 1-6 heteroatom(s) selected from the group consisting of O, N, and S. The substituents attached to a phenyl ring portion of an aryl moiety in the compounds of the invention may be configured in the ortho-, meta-, or para-orientation(s), with the para-orientation being preferred.

Examples of typical aryl moieties included in the scope of the present invention may include, but are not limited to, the following:

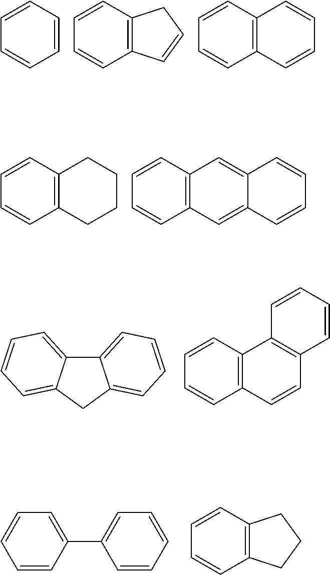

It should be kept in mind that, throughout this application, “Ar” is used to designate various substituents. As indicated throughout, these Ar groups are independently selected. Thus, for example, the fact that Ar may be phenyl in one context does not require that Ar be phenyl, nor prohibit that Ar be, for example, pyridyl in another context in the same molecule. It is intended that all “Ar” are selected independently of all other “Ar”, whether or not the term “independently selected” is used.

“Carbocycle” or “carbocyclic” refers to an organic cyclic moiety in which the cyclic skeleton is comprised of only carbon atoms, whereas the term “heterocycle” or “heterocyclic” refers to an organic cyclic moiety in which the cyclic skeleton contains one or more heteroatoms selected from nitrogen, oxygen, or sulfur, and which may or may not include carbon atoms. The term “carbocycle” refers to a carbocyclic moiety containing the indicated number of carbon atoms. The term “C₃-C₈ cycloalkyl”, therefore, refers to an organic cyclic substituent in which three to eight carbon atoms form a three, four, five, six, seven, or eight-membered ring, including, for example, a cyclopropyl, cyclobutyl, cyclopentyl, cyclohexyl, cycloheptyl, or cyclooctyl ring.

“Carbocyclic” or “heterocyclic” each includes within its scope a single ring system, multiple fused rings (for example, bicyclic, tricyclic, or other similar bridged ring systems or substituents, e.g. adamantyl) or multiple condensed ring systems. One skilled in the art, therefore, will appreciate that in the context of the present invention, a cyclic structure may comprise bi-, or tri-, or multiple condensed rings, bridged ring systems, or combinations thereof.

“Halo” refers to fluoro, chloro, bromo or iodo, unless otherwise indicated.

“Heterocycle” or “heterocyclic”, refers to a saturated, unsaturated, or aromatic carbocyclic group having a single ring, multiple fused rings (for example, bicyclic, tricyclic, or other similar bridged ring systems or substituents), or multiple condensed rings, and having at least one heteroatom such as nitrogen, oxygen, or sulfur within at least one of the rings. This term also includes “Heteroaryl,” which refers to a heterocycle in which at least one ring is aromatic. Any heterocyclic or heteroaryl group can be unsubstituted or optionally substituted with one or more groups, as defined above. Further, bi- or tricyclic heteroaryl moieties may comprise at least one ring which is either completely or partially saturated.

As one skilled in the art will appreciate, such heterocyclic moieties may exist in several isomeric forms, all of which are encompassed by the present invention. For example, a 1,3,5-triazine moiety is isomeric to a 1,2,4-triazine group. Such positional isomers are to be considered within the scope of the present invention. Likewise, the heterocyclic or heteroaryl groups can be bonded to other moieties in the compounds of the present invention. The point(s) of attachment to these other moieties is not to be construed as limiting on the scope of the invention. Thus, by way of example, a pyridyl moiety may be bound to other groups through the 2-, 3-, or 4-position of the pyridyl group. All such configurations are to be construed as within the scope of the present invention.

Examples of heterocyclic or heteroaryl moieties included in the scope of the present invention may include, but are not limited to, the following:

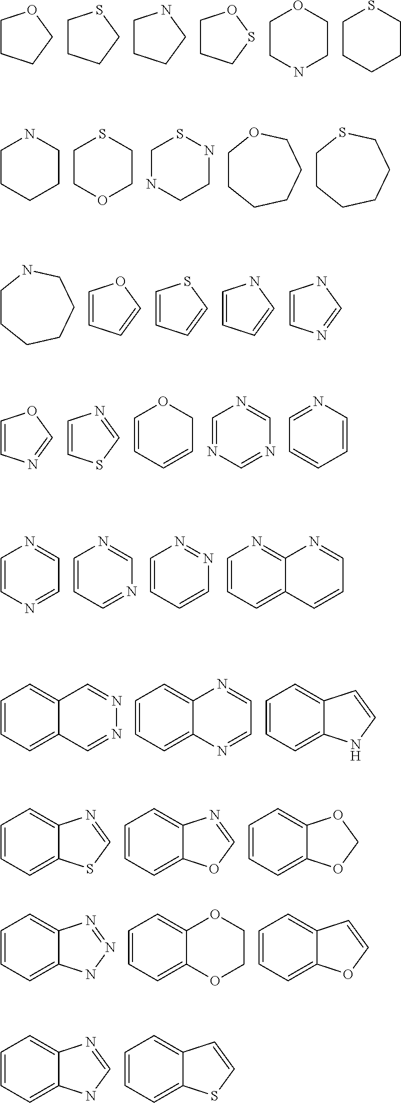

### Methods of the Present Invention

DNA associated with histone proteins is organized into nucleosomes, which are then arranged into higher-order structures known as chromatin. The N-terminal tails of histone proteins are the sites of many different types of modifications, including acetylation, phosphorylation, ubiquitination, and methylation. Acetylation of the ε-amino group of specific lysine residues achieved by histone acetyltransferase enzymes typically results in transcriptional activation and gene expression. On the other hand, hypoacetylation mediated by histone deacetylase enzymes is associated with repression of gene expression and the maintenance of transcriptionally silent chromatin.

In addition to deacetylating histone tails, transcription factors such as p53, GATA-1, TFIIE, and TFIIF are HDAC substrates. HDACs participate in cell cycle control and growth regulation, and consequently inhibitors of HDACs have demonstrated the ability to arrest tumor cell growth, induce differentiation, and cause apoptosis. A growing body of experimental evidence indicates that the activity of HDAC affects cell cycle arrest, terminal differentiation of different cell types, and the pathogenesis of malignant disease. HDACs play integral roles in cancer gene regulation and cancer proliferation and HDAC inhibitors represent promising new chemotherapies. For example, the role of HDAC has been highlighted by the finding that mutant retinoid receptors recruit HDAC in acute promyelocytic leukemia. Thus, a thorough characterization of HDAC activity is important not only to our understanding of gene expression, but to our understanding of cancer pathology.

Interestingly, HDAC inhibitors such as trichostatin A and trapoxin have been shown to induce cell differentiation, cell cycle arrest, and reversal of transformed cell morphology. Not surprisingly, therefore, a number of HDAC inhibitors show promise as antitumor agents. The discovery of antimalarial effects of certain HDAC inhibitors further supports the idea that HDACs will be key targets for chemotherapeutic intervention in a variety of human diseases.

Histone deacetylases regulate chromatin remodeling and transcriptional silencing by removing acetyl groups from conserved lysine residues located on the N-terminal tails of histones. It is believed that histone deacetylases interact with, and are recruited by tumor suppressors and oncogenes. As a result, natural product inhibitors of the histone deacetylase function, such as sodium butyrate and hydroxamic acids, are expected to be effective therapeutic agents for the treatment of human cancers. Characterization of HDAC activity has previously been problematic due to a lack of pure, active enzyme and a suitable activity assay.

Fluorescence-based biological assays have been widely used for high-throughput screening for inhibitors of pharmaceutically interesting target enzymes. Suitable assay formats, however, have not yet been developed for all important targets. For HDACs, the most frequently used assay type is still isotopic and nonhomogeneous in nature. Homogeneous fluorogenic assays are highly desirable because a reaction product is released as a fluorescent moiety, distinguishable from the reaction substrate, during the enzymatic reaction.

The inventive subject matter provides in vitro monitoring of HDAC activity via fluorogenic assay. The inventive HDAC substrates contain an acetylated coumarin moiety which is optionally substituted. We have synthesized an exemplary compound, 7-Acetoxycoumarin (hereinafter “7AC”), and employed it as a small molecule substrate for continuous monitoring of HDAC activity in the first continuous fluorescent assay for histone deacetylase activity. The inventive assay is nonradioactive, highly sensitive, and does not demand the consumption of expensive material such as histones. Experiments with Trichostatin A and sodium butyrate, known inhibitors of HDAC, indicate that the new assay is well suited for high-throughput screening efforts for identifying novel HDAC inhibitors from collections of candidate compounds and compositions.

It is expected that the inventive subject matter will greatly streamline the process of selecting HDAC inhibitors and make more feasible the identification of inhibitors targeted to specific HDACs. Thus, inhibition of a specific HDAC within an organism is expected to make possible the targeted inhibition of HDAC enzyme(s) responsible for a specific disorder, such as an uncontrolled proliferation disorder. Similarly, inhibition of an HDAC of a specific organism is expected to make possible the targeted inhibition of that organism, such as is found in a protozoal or fungal infection.

### Method for Screening for Histone Deacetylase Activity

The present invention relates to a method for screening for histone deacetylase enzyme activity in a test sample, comprising the steps of:

i) contacting said test sample with a liquid screening solution comprising a compound of Formula I:

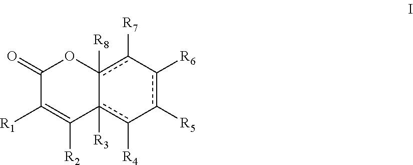

wherein:

R₁, R₂, R₃, R₄, R₅, R₆, R₇, and R₈ are independently selected from the group consisting of:

hydrogen,

hydroxyl,

carbonyl,

acetyl,

Ar,

straight or branched chain C₁-C₉ alkyl,

straight or branched chain C₁-C₉ alkyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

straight or branched chain C₂-C₉ alkenyl or alkynyl, and

straight or branched chain C₂-C₉ alkenyl or alkynyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

provided that at least one of R₁, R₂, R₃, R₄, R₅, R₆, R₇, and R₈ must be —O—C(O)—R₈, wherein R₉ is straight or branched chain C₁-C₃ alkyl; and

Ar is a mono-, bi- or tricyclic, carbo- or heterocyclic ring, wherein the ring is either unsubstituted or substituted in one or more position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, or amino; wherein the individual ring sizes are 5-6 members; and wherein the heterocyclic ring contains 1-6 heteroatom(s) selected from the group consisting of O, N, and S; and

ii) assaying for fluorescence produced by a product of histone deacetylase enzyme activity in said liquid screening solution.

In another aspect of the inventive subject matter, said Ar is selected from the group consisting of naphthyl, indolyl, thioindolyl, furyl, thiazolyl, thienyl, pyridyl, quinolinyl, isoquinolinyl, fluorenyl, phenyl, and benzyl.

In a preferred embodiment, said Ar is phenyl.

In another aspect of the inventive subject matter, R₆ is —O—C(O)—R₉.

In a preferred embodiment, said R₆ is acetyl.

In a more preferred embodiment, said R₁, R₂, R₃, R₄, R₅, R₇, and R₈ are independently selected from the group consisting of:

hydrogen,

hydroxyl,

carbonyl,

acetyl,

Ar,

straight or branched chain C₁-C₃ alkyl, and

straight or branched chain C₁-C₈ alkyl substituted with halo, trifluoromethyl, nitro, C₁-C₈ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

In another more preferred embodiment, said R₁, R₂, R₃, R₄, R₅, R₇, and R₈ are independently selected from the group consisting of:

hydrogen,

hydroxyl,

carbonyl,

acetyl,

Ar,

straight or branched chain C₁-C₃ alkyl, and

straight or branched chain C₁-C₃ alkyl substituted with halo, trifluoromethyl, nitro, hydroxy, phenoxy, benzyloxy, amino, or Ar.

In a further more preferred embodiment, said R₁, R₂, R₃, R₄, R₅, R₇, and R₈ are independently selected from the group consisting of:

hydrogen,

methyl,

hydroxyl,

carbonyl,

acetyl, and

phenyl.

In another aspect of the inventive subject matter, R₁, R₂, R₃, R₄, R₅, R₆, R₇, and R₈ are independently selected from the group consisting of:

hydrogen,

hydroxyl,

carbonyl,

acetyl,

Ar,

straight or branched chain C₁-C₃ alkyl, and

straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

In a particularly preferred embodiment, said compound is of Formula II:

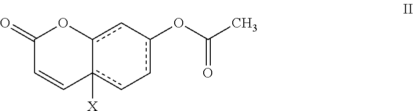

wherein X is selected from the group consisting of hydrogen, methyl, hydroxyl, carbonyl, acetyl, and phenyl.

In a most preferred embodiment, X is hydrogen or phenyl, and said assay for fluorescence is conducted at about 447 nm.

### Method for Screening for Histone Deacetylase Inhibitors

The present invention further relates to a method for screening for inhibitors of histone deacetylase enzyme activity, comprising the steps of:

i) contacting an inhibitor candidate compound or composition with a liquid screening solution comprising a histone deacetylase enzyme and a compound of Formula I:

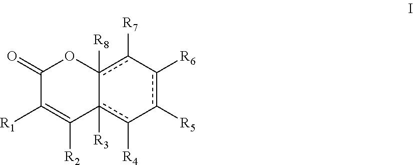

wherein:

R₁, R₂, R₃, R₄, R₅, R₆, R₇, and R₈ are independently selected from the group consisting of:

hydrogen,

hydroxyl,

carbonyl,

acetyl,

Ar,

straight or branched chain C₁-C₃ alkyl,

straight or branched chain C₁-C₉ alkyl substituted with one or more halo, trifluoromethyl, nitro, C₁-C₈ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

straight or branched chain C₂-C₉ alkenyl or alkynyl, and

straight or branched chain C₂-C₉ alkenyl or alkynyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,

provided that at least one of R₁, R₂, R₃, R₄, R₅, R₆, R₇, and R₈ must be −0-C(O)—R₈, wherein R₉ is straight or branched chain C₁-C₃ alkyl; and

Ar is a mono-, bi- or tricyclic, carbo- or heterocyclic ring, wherein the ring is either unsubstituted or substituted in one or more position(s) with halo, hydroxyl, nitro, trifluoromethyl, C₁-C₈ straight or branched chain alkyl or alkenyl, C₁-C₄ alkoxy, C₁-C₄ alkenyloxy, phenoxy, benzyloxy, or amino; wherein the individual ring sizes are 5-6 members; and wherein the heterocyclic ring contains 1-6 heteroatom(s) selected from the group consisting of O, N, and S; and

ii) assaying for fluorescence produced by a product of histone deacetylase enzyme activity in said reaction solution.

In another aspect of the inventive subject matter, said Ar is selected from the group consisting of naphthyl, indolyl, thioindolyl, furyl, thiazolyl, thienyl, pyridyl, quinolinyl, isoquinolinyl, fluorenyl, phenyl, and benzyl.

In a preferred embodiment, said Ar is phenyl.

In another aspect of the inventive subject matter, R₆ is —O—C(O)—R₉.

In a preferred embodiment, said R₆ is acetyl.

In a more preferred embodiment, said R₁, R₂, R₃, R₄, R₅, R₇, and R₈ are independently selected from the group consisting of:

hydrogen,

hydroxyl,

carbonyl,

acetyl,

Ar,

straight or branched chain C₁-C₃ alkyl, and

straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

In another more preferred embodiment, said R₁, R₂, R₃, R₄, R₅, R₇, and R₈ are independently selected from the group consisting of:

hydrogen,

hydroxyl,

carbonyl,

acetyl,

Ar,

straight or branched chain C₁-C₃ alkyl, and

straight or branched chain C₁-C₃ alkyl substituted with halo, trifluoromethyl, nitro, hydroxy, phenoxy, benzyloxy, amino, or Ar.

In a further more preferred embodiment, said R₁, R₂, R₃, R₄, R₅, R₄, and R₈ are independently selected from the group consisting of:

hydrogen,

methyl,

hydroxyl,

carbonyl,

acetyl, and

phenyl.

In another aspect of the inventive subject matter, R₁, R₂, R₃, R₄, R₅, R₆, R₇, and R₈ are independently selected from the group consisting of:

hydrogen,

hydroxyl,

carbonyl,

acetyl,

Ar,

straight or branched chain C₁-C₃ alkyl, and

straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

In a particularly preferred embodiment, said compound is of Formula II:

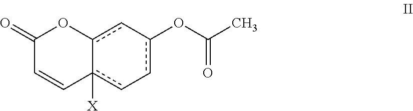

wherein X is selected from the group consisting of hydrogen, methyl, hydroxyl, carbonyl, acetyl, and phenyl.

In a most preferred embodiment, X is hydrogen or phenyl, and said assay for fluorescence is conducted at about 447 nm.

The novel HDAC assay presented herein combines the specificity of the deacetylation reaction with the advantages of a homogeneous fluorogenic assay in a single-step process. The inventive HDAC substrates produce a de-acetylated fluorophore which is detectable by standard fluorescence measurements. The assay described here is well suited in the context of high-throughput screening for HDAC inhibitors, even in small reaction volumes.

It will be understood by one of ordinary skill in the art that enzymes having HDAC activity generally function in coordination complexes additionally comprising one or more divalent metal ion(s), particularly zinc. Thus, in the case of a recombinant enzyme for example, it may be necessary for a sample to be tested for HDAC activity, or a screening solution for detecting inhibition of HDAC activity, to additionally comprise divalent metal ions. Exemplary divalent metal ions, which may be required to form functional HDAC enzyme-metal coordination complexes include, but are not limited to, zinc, copper, manganese, iron, nickel, calcium, and magnesium. Zinc and manganese are the more preferred among such exemplary divalent metal ions. We have found that manganese is particularly effective with the Hos3 enzyme.

Spectrophotomeric HDAC Activity Assays. Three different chromophores were utilized in developing the inventive subject matter:

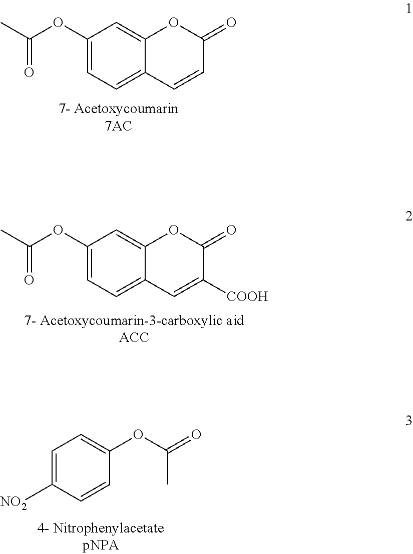

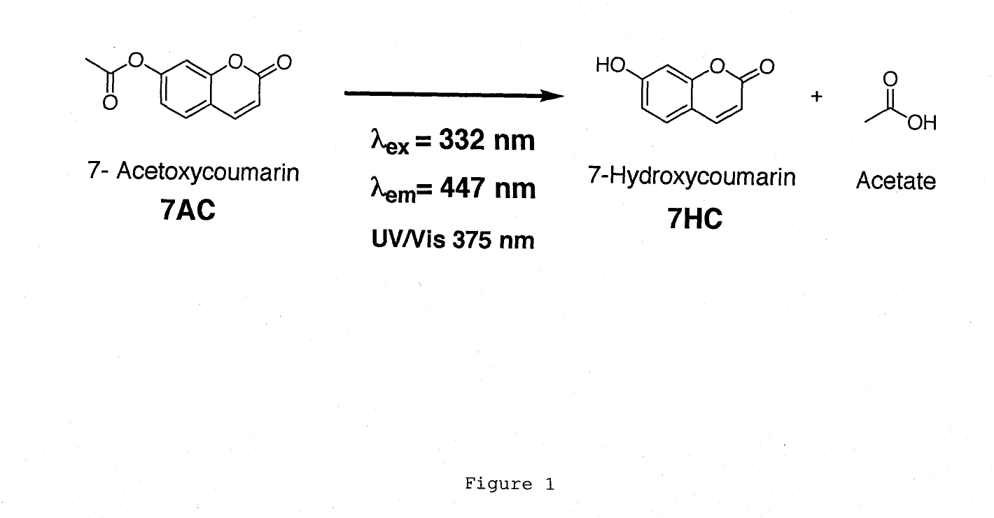

As depicted in FIG. 1, the inventive fluorescence-based HDAC assay monitors the hydrolysis of 7AC to 7-hydroxycoumarin, or of ACC to 7-hydroxycoumarin-3-carboxylic acid, at λₑₓ=332 nm and λₑₘ=447 nm. 7AC has an advantage over ACC due to a lower background rate of hydrolysis in the assay buffer at pH 8.0. Utilization rations for each enzyme with the 7AC substrate were calculated. For Hos3, the utilization ratio was 1.9122×10⁻⁴ s⁻¹, and 1274.8 M⁻¹ s⁻¹ when normalized by enzyme concentration. For HDLP, the utilization ratio was 2.0375×10⁻⁴ s⁻¹, and 612.8 M⁻¹ s⁻¹ when normalized by enzyme concentration. For HDAC2, the utilization ratio was 1.2526×10⁻⁵ s⁻¹, and 35.8 M⁻¹ s⁻¹ when normalized by enzyme concentration, as shown in Table 1.

TABLE 1
Utilization Ratios Determined Using 7AC Substrate

| Enzyme | V/K (s⁻¹) | V/[Eₜ]/K (M⁻¹ s⁻¹) |
|---|---|---|
| Hos3 | 1.9122 × 10⁻⁴ | 1274.8 |
| HDLP | 2.0375 × 10⁻⁴ | 612.8 |
| HDAC2 | 1.2526 × 10⁻⁵ | 35.8 |

The substrates 7AC and ACC can also be effectively utilized in a UV assay. The substrate absorbs from 250-340 nm, while the product has a maximal absorbance at 375 nm. So substrate hydrolysis/product formation can be monitored by the increase in absorbance at 375 nm. For Hos3, the Kₘ^{app} with 7AC using the UV assay was 649 mM. The Kₘ^{app} may be lower than the actual Kₘ due to the limited solubility of the substrate in the assay buffer at concentrations greater than 1 mM. Para-nitrophenylacetate can also be used to assay HDAC activity. Upon acetate hydrolysis, the para-nitrophenylacetate liberates the colored p-nitrophenol chromophore; this reaction can be monitored on the UV at 410 nm. Using MBP-Hos3, the Kₘ^{app} was determined to be 8.3 mM. This substrate is also subject to solubility limitations that prevent complete saturation of the enzyme.

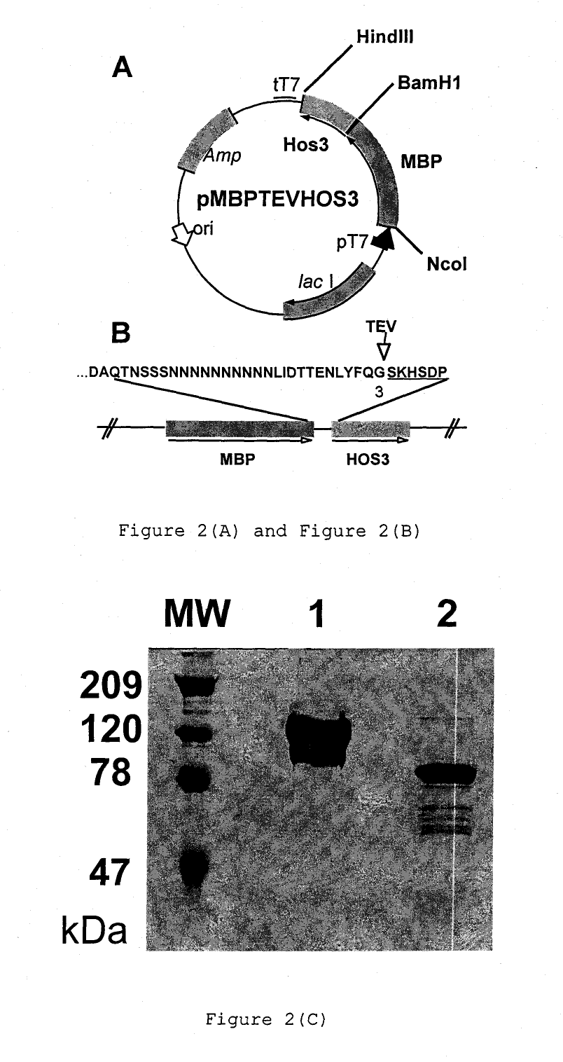

Protein Expression and Purification. FIG. 2A shows MBP-Hos3 contains an internal TEV protease site for select proteolytic removal of the MBP fusion protein. Similarly, FIG. 2B shows an expansion of the junction between Hos3 and MBP showing the TEV protease cleavage site. As demonstrated in FIGS. 2A and 2B, the Hos3 gene was successfully cloned into the pMBP parallel2 vector to produce pMBPTEVHOS3. The MBP fusion protein was then overexpressed in E. coli BL21 (DE3) cells. As shown in FIG. 2C, purification resulted in a final yield of approximately 20 mg Hos3 protein per liter cells. In FIG. 2C, lane 1: Affinity-purified MBP-Hos3; lane 2: Heparin-agarose purified Hos3; remaining lane: molecular weight standards.

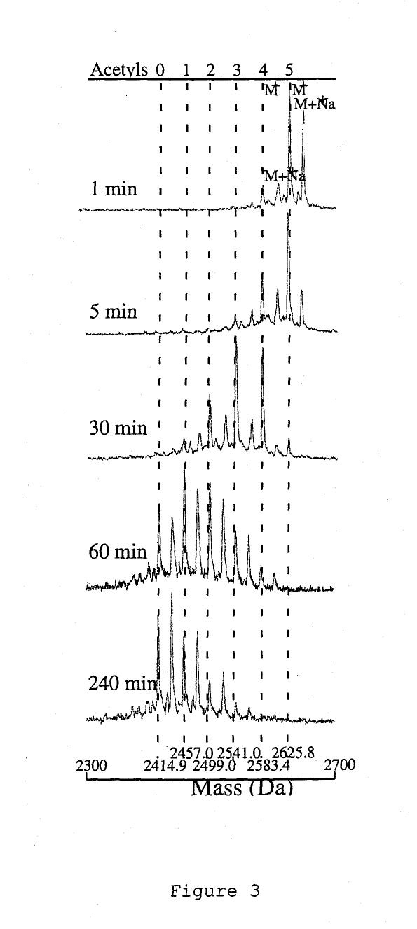

Characterization of Hos3 Activity by Mass Spectrometry. Hos3 demonstrated the ability to deacetylate a hyperacetylated histone H4(1-23) substrate in vitro. 1 uM MBP-Hos3 enzyme was incubated with 100 uM of histone H4 (1-24) acetylated peptide substrate for 240 minutes in buffer A. Aliquots removed during the time course were subjected to MALDI-TOF mass spectrometry for mass determination. Aliquots were removed at specific time points and quenched with 0.01% TFA and desalted by HPLC. As shown in FIG. 3, all acetylated positions, Lys 5, 8, 12, 16, and 20 were deacetylated by MBP-Hos3.

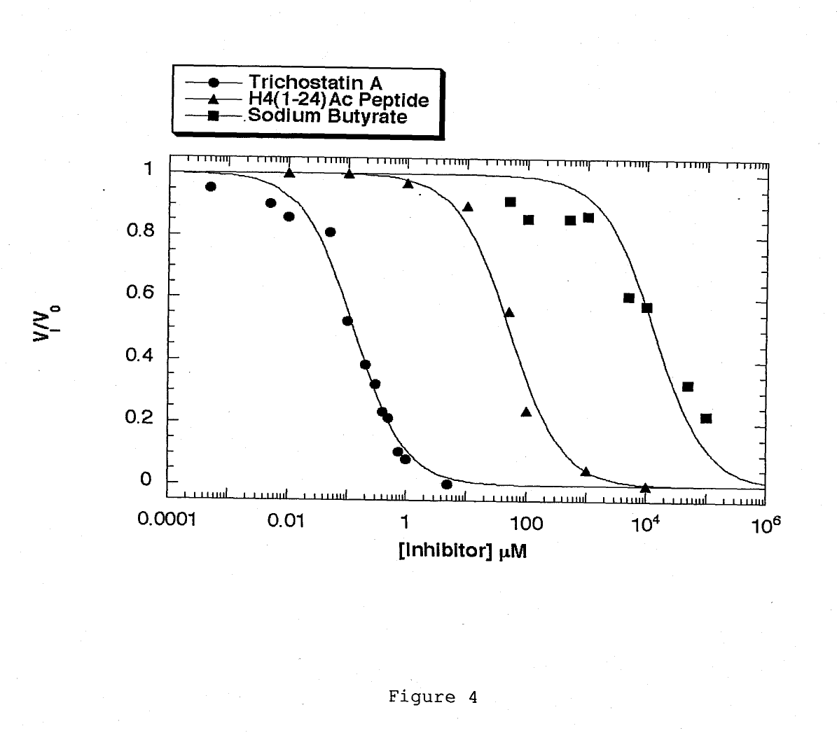

Characterization of the Mechanism of Inhibition. Inhibitor studies were conducted using the 7AC substrate and the fluorescent histone deacetylase activity assay. As shown in Table 2 and FIG. 4, IC₅₀ values of Hos3 (150 nM) with 7AC (75 mM) substrate and alternate substrate histone H4 (1-24) acetylated peptide, and inhibitors Trichostatin A and sodium butyrate, were determined using our fluorescent assay. The mixed-competitive inhibitor sodium butyrate displayed an IC₅₀ of 13.4 (±3.8) mM and a Kᵢ of 12 mM.

TABLE 2

| | | Mode of | | | | |
|---|---|---|---|---|---|---|
| | | Inhibition | IC₅₀ value | | Kᵢ | |
| H4(1-24) | Alternate | Non- | 51.2 ± 7 | μM | 50 | μM |
| | Substrate | Competitive | | | | |
| NaB | Inhibitor | Mixed- | 13.4 ± 3.8 | mM | 12 | mM |
| | | Competitive | | | | |
| TSA | Inhibitor | Tight-binding | 127.0 ± 11.0 | nM | 81 | nM |
| | | Comp | | | | |

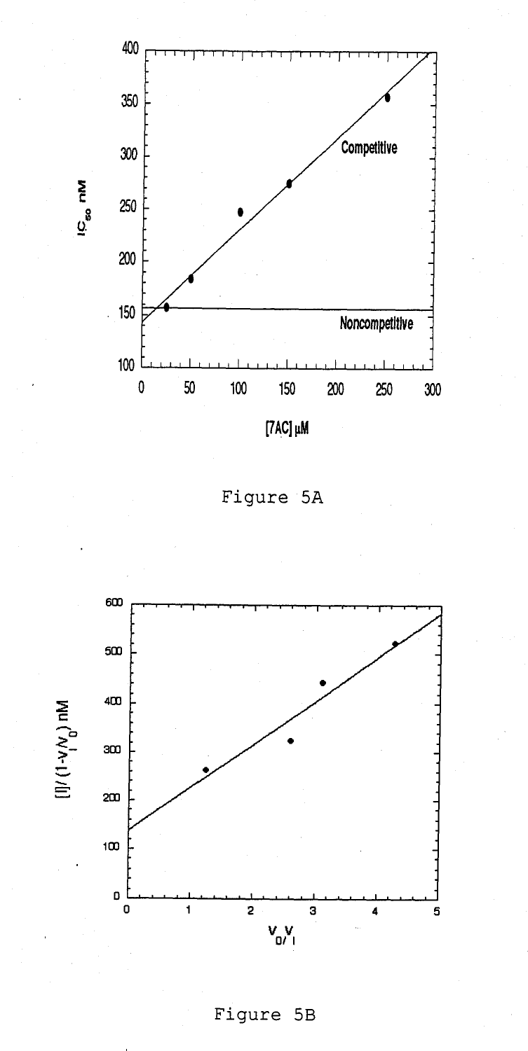

Despite showing apparent non-competitive inhibitory patterns, Trichostatin A was proven to be a tight-binding competitive inhibitor when IC₅₀ values were plotted as a function of substrate concentration, as shown in FIG. 5A. The Kᵢ=81 nM was obtained from the slope of the line of the Henderson plot depicted in FIG. 5B. AS shown in Table 2 and FIGS. 5A and 5B, Trichostatin A is a tight-binding competitive inhibitor with an IC₅₀ value of 127.0 (±11.0) nM and a Kᵢ of 81 nM.

Characterization of an Alternate Substrate. Either spectrophotomeric HDAC assay can be used to screen alternate substrates by competition. As shown in Table 2, the IC₅₀ and Kᵢ values for alternate substrate histone H4(1-24)Ac were determined to be 51.2 (±7) μM and 50 μM, respectively.

Using the inventive substrates, kinetic parameters and utilization ratios for each of three representative Class I/II zinc-dependent HDAC enzymes, Saccharomyces cerevisiae Hos3, human HDAC2, and Aquifex aeolicus HDLP, were determined. Using 7AC, we determined that Trichostatin A, a hydroxamate inhibitor that previously was thought to exhibit non-competitive inhibitory patterns with acetylated histones, is actually a tight-binding, competitive inhibitor with a Kᵢ value of 81 nM and an IC₅₀ value of 127±11 nM. Sodium butyrate displays mixed-type inhibition with a Kᵢ value of 12 mM and an IC₅₀ of 13.4±3.8 mM. Hos3 exhibited non-competitive inhibition with alternate substrate acetylated histone peptide H4 (1-24) with a Kᵢ value of 50 mM and an IC₅₀ value of 51.2±7 μM. Based on these studies, we expect broad utility of using acetoxycoumarins as fluorogenic substrates for assaying for HDAC activity. The discovery of a small molecule fluorescent substrate for HDACs now makes more feasible the design and high-throughput evaluation of inhibitors for identification of cancer chemotherapeutic agents.

### Synthesis of Compounds of the Invention

The inventive compounds may be readily prepared by standard techniques of organic chemistry. In the preparation of the compounds of the invention, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to “Protective Groups in Organic Chemistry,” McOmie, ed., Plenum Press, New York, N.Y.; and “Protective Groups in Organic Synthesis,” Greene, ed., John Wiley & Sons, New York, N.Y. (1981) for the teaching of protective groups which may be useful in the preparation of compounds of the present invention.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

### EXAMPLES

The following examples are illustrative of the present invention and are not intended to be limitations thereon. Unless otherwise indicated, all percentages are based upon 100% by weight of the final composition.

DNA contained in the plasmid pHOS3.S2 plasmid DNA was generously provided by Prof. Michael Grunstein from University of California at Los Angeles. Human HDAC2 clone as the GST-HDAC2 was generously provided by Prof. Edward Seto from the University of Florida. GST-HDLP was generously provided by Prof. Nikola Pavletich at Memorial Sloan Kettering Cancer Center. PMBP-parallel 2 vector was obtained from Prof. Patrick Loll at Drexel University. Dideoxy DNA sequencing was performed by the University of Pennsylvania Molecular Biology DNA Sequencing Core Facility.

Oligonucleotide primers, electrocompetent E. coli BL21 (DE3) cells, tobacco etch virus (TEV) protease, ampicillin, heparin agarose, glutathione, Luria broth, isopropyl-β-D-thiogalactopyranoside, amylose resin, glutathione sepharose, Trichostatin A, 7-acetoxycoumarin-3-carboxylic acid, and para-nitrophenylacetate were obtained from publicly available, commercial sources and are known to those of ordinary skill in the art. Other reagents and solvents were purchased from publicly available commercial sources and used without further purification.

Abbreviations used in the Examples are the following: “HDAC” refers to histone deacetylase; “HAT” refers to histone acetyl transferase; “Ac” refers to acetyl; “ACC” refers to 7-acetoxycoumarin-3-carboxylic acid; “7AC” refers to 7-acetoxycoumarin; “pNPA” refers to 4-nitrophenylacetate; “TSA” refers to Trichostatin A; “IPTG” refers to isopropyl-b-D-thiogalactoside; “MBP” refers to maltose-binding protein; “GST” refers to glutathione-s-transferase; “LB” refers to Luria-Burtani media; “PCR” refers to polymerase chain reaction; “SDS” refers to sodium dodecyl sulfate; “PAGE” refers to polyacrylamide gel electrophoresis; “DMSO” refers to dimethylsulfoxide; “Kₘ” refers to Michaelis constant; “k_{cat}” refers to catalytic turnover number; “K_{I}” refers to the dissociation constant of the inhibitor; “IC₅₀” refers to 50% inhibitory concentration; “ESI-FTMS” refers to high resolution Fourier transform electrospray mass spectrometry; “MALDI-TOF” refers to matrix assisted laser desorption ionization time-of-flight mass spectrometry; “TEV” refers to tobacco etch virus.

### Example 1

### Synthesis of 7-Acetoxycoumarin (7AC)

The following example illustrates the preparation of a preferred HDAC substrate provided according to the present inventive subject matter.

To an ice cold solution of 20% acetic anhydride in pyridine (60 ml) was added 25 mmol of 7-hydroxycoumarin (Aldrich) with stirring so the temperature did not rise above 4° C. The reaction immediately darkened, stirring was continued at 0° C. for 30 min, the reaction was allowed to warn to room temperature, and stirred an additional 2.5 h. The reaction mixture was rotary evaporated to dryness and the residue was taken up in 100 ml EtOAc and washed with 10% citric acid (5×100 ml), brine (5×100 ml) dried with MgSO₄ and evaporated to dryness. The resulting solid was recrystallized two times from EtOAc/hexanes to provide the title compound as a white solid: m.p. 135-137° C.; ¹H NMR d (ppm) 2.3 (s, 3H, CH₃), 6.4 (d, 1H, J=9.5 Hz, H2′), 7.1 (dd, 2H, J=1.5 Hz, J=8.5 Hz, H3′, H2) 7.2 (d, 2H, J=1.5 Hz, H-2) 7.7 (d, 2H, J=9 Hz, H-5), 8.0 ppm (d, 2H, J=9.5 Hz, H-6); ¹³C NMR δ=20.721, 109.969, 115.395, 116.512, 118.504, 129.188, 143.647, 152.777, 153.967, 159.588, 168.670 ppm.

### Example 2

### Cloning, Overexpression and Purification of Hos3

Using PCR oligonucleotide primers derived from the gene sequence, the Saccharomyces cerevisiae Hos3 gene from pHOS3.S2 plasmid DNA was subcloned into the Bam HI/HindIII sites of plasmid MBP-2 of pMBP parallel 2 to produce pMBPTEVHOS3. This vector is a T7 promoter-based expression vector that produces Hos3 N-terminally fused to MBP, with an intervening TEV protease recognition site for fusion protein removal after affinity purification. The integrity of the clone was established by DNA sequencing. The pMBPTEVHOS3 plasmid was subsequently transformed into BL21(DE3) E. coli electrocompetent cells for overexpression. Bacterial cultures were grown in LB media containing 200 μg/ml ampicillin to an optical density of 0.8 at 1=595 nM. Cells were induced with 1 mM IPTG and grown for 4 hours at 37° C. with shaking (200 rpm) then harvested by centrifugation (3000×g, 4° C., for 10 minutes.) Pellets from 2 L cells were resuspended in Buffer A (20 mM Tris, pH 8.0 and 200 mM NaCl), lysed in a French Pressure cell and centrifuged at 150,000×g, 4° C., for 1 h. The supernatant was diluted to 50 ml and applied to amylose resin (1 ml/min) previously equilibrated with Buffer A. The MBP fusion protein was eluted from the amylose with Buffer B (20 mM Tris, pH 8.0 and 200 mM NaCl, 10 mM maltose). Fractions containing the desired protein were identified by SDS-PAGE (12% acrylamide), combined, and dialyzed overnight into Buffer A. The protein was adjusted to 2.0 mg/ml and the fusion tag was removed proteolytically by the addition of TEV protease (1:8 dilution) for 5 hours at room temperature. The proteolysis reaction mixture was applied to heparin agarose, previously equilibrated with Buffer A. The resin was washed in Buffer A (3×10 ml) then cut Hos3 was eluted with Buffer C (20 mM Tris, pH 8.0 and 500 mM NaCl). Purified Hos3 was confirmed by SDS-PAGE. Protein concentrations were determined using the von Hippel method. Protein aliquots were then flash frozen in liquid nitrogen and stored at −80° C. until use.

### Example 3

### Overexpression and Purification of HDAC2 and HDLP

E. coli BL21 (DE3) cells harboring the GST-HDAC2 and GST-HDLP expression plasmids were grown, expressed, and harvested in the same manner as was performed for pMBPTEVHOS3. Cell pellets were resuspended in Buffer A, lysed in a French Pressure cell, and centrifuged at 150,000 4° C., for 1 hour. The supernatant was diluted with Buffer A and incubated with glutathione sepharose beads (10 ml) for 1 hour, loaded on to a gravity filtration column, and washed with 10 column volumes of Buffer A. GST fusion proteins were eluted with Buffer D (20 mM Tris, pH 8.0, 200 mM NaCl, 10 mM reduced glutathione). Fractions containing purified GST proteins identified by SDS-PAGE were combined and dialyzed overnight into Buffer A. GST-HDAC2 and GST-HDLP were concentrated to 0.1 and 2.3 mg/ml respectively, aliquoted, flash frozen in liquid nitrogen, and stored at −80° C.

### Example 4

### Hos3 Deacetylation of Hyperacetylated Histone H4(1-23)

The histone H4(1-23) peptide was synthesized by standard FMOC solid-phase peptide synthesis methods and hyperacetylated at Lys 5, 8, 12, 16, and 20 post-cleavage with acetic anhydride in DMF/triethylamine. To confirm that Hos3 exhibited histone deacetylase activity, we incubated the peptide (100 μM) for 240 minutes with MBP-Hos3 (1 μM enzyme); aliquots removed throughout the time course, desalted by HPLC and analyzed by matrix-assisted time of flight mass spectrometry (MALDI TOF-MS). The starting hyperacetylated peptide mass was 2625.8 Da, and for each loss of an acetyl group the mass decreased by approximately 42 Da.

### Example 5

### Histone Deacetylase Fluorescent Activity Assay

HDAC activity assays were performed in HDAC assay buffer (20 mM Tris, pH 8.0, 500 mM NaCl, 1 mM MnCl₂) to a final reaction volume of 1 ml in quartz spectrophotometer cuvettes. Fluorescence spectrophotometry was used to monitor ACC or 7AC hydrolysis (λₑₓ=332 nm, λₑₘ=447 nm). Internal fluorescence intensity calibrations were obtained using quinine sulfate as known in the art. ACC or 7AC substrate (solubilized in 100% DMSO) was added in the appropriate concentrations to the assay buffer (1% final concentration of DMSO) and the basal rate of hydrolysis was recorded. After 90 seconds, 150 nM Hos3 enzyme was added and the reaction was allowed to proceed for 600 seconds. Initial rates for Hos3 activity were defined as the initial rate of the reaction with enzyme added, minus the basal rate of substrate hydrolysis. Utilization ratios for Hos3 were calculated based on the adjusted initial rates. The data was fit by the following expressions (equations 1-3):

v=Vₘₐₓ[S]/Kₘ+[S], where [S]<<Kₘ (1)

giving v=Vₘₐₓ[S]/Kₘ (2)

which rearranges to v/[S]=Vₘₐₓ/Kₘ (3)

where Vₘₐₓ is the maximum reaction velocity, [S] is the substrate concentration, Kₘ is the Michaelis constant, and Vₘₐₓ/Kₘ is the utilization ratio (V/K). Since V is dependent on the total concentration of enzyme [Eₜ], utilization ratios are normalized by enzyme concentration and reported in terms of V/[Eₜ]/K. For GST-HDLP and GST-HDAC2, assay conditions and procedures were the same except 330 nM of GST-HDLP and 350 nM of GST-HDAC2 were added to each respective reaction.

### Example 6

### UV-Based Histone Deacetylase Activity Assays

HDAC activity assays were performed in HDAC Assay Buffer to a final reaction volume of 1 ml in quartz cuvettes. UV/Vis spectrophotometer was used to monitor the rate of 7AC hydrolysis. The 7AC substrate absorbs from 250-340 nm, while the product, 7-hydroxycoumarin, has a maximal absorbance at 375 nm. Reactions were initiated with substrate and allowed to proceed for 300 seconds, while monitoring the increase in absorbance at 375 nm. Para-nitrophenylacetate was also used to assay HDAC activity. Upon acetate hydrolysis, the para-nitrophenylacetate liberated the colored p-nitrophenol chromophore; this reaction was monitored on the UV at 410 nm. Initial rates and utilization ratios were calculated as described above.

### Example 7

### HDAC Inhibitor Studies

Competition experiments were performed in HDAC assay buffer (20 mM Tris, pH 8.0, 500 mM NaCl, 1 mM MnCl₂) to a final reaction volume of 1 ml. Trichostatin A was solubilized in 100% DMSO, yielding a final concentration of 2% DMSO in this reaction. Sodium butyrate and H4 (1-24) Ac peptide were both dissolved in water. The final DMSO concentration in these reactions was 1%. 7AC substrate and inhibitor were added in the appropriate concentrations to the assay buffer and the basal rate of hydrolysis was recorded. Hos3 enzyme (150 nM) was added after 90 seconds and the reaction was allowed to proceed for 600 seconds. Initial rate data was transformed into appropriate rates and dose response plots were generated. IC₅₀ values were calculated using the program Kaleidagraph with equation 4:

vᵢ/vₒ=1/1+([I]/IC₅₀) (4)

where vᵢ is the initial velocity in the presence of inhibitor, vₒ, is the initial velocity in the absence of inhibitor, and [I] is the concentration of inhibitor. Kᵢ values were determined by Dixon plots (ref). The modes of inhibition were determined by double-reciprocal or Lineweaver-Burk plots, where 1/v is plotted as a function of 1/[S], using the programs of Cleland.

In the case of Trichostatin A, the double-reciprocal plot yielded apparent non-competitive inhibition that was later determined to be tight binding competitive inhibition by plotting IC₅₀ values as a function of [S] substrate concentration, which resulted in a straight line with a positive slope. To get the Kᵢ value, points between the range of vᵢ/vₒ=0.2 to 0.9 from the dose-response curve were used to generate a Henderson plot, where [I]/(1-vᵢ/vₒ) is plotted as a function of vᵢ/vₒ, with the slope of the line equal to Kᵢ (1+[S]/Kₘ) and the y-intercept equal to the total enzyme concentration [Eₜ].

The invention being thus described, it will be obvious that the same may be modified or varied in many ways. Such modifications and variations are not to be regarded as a departure from the spirit and scope of the invention and all such modifications and variations are intended to be included within the scope of the following claims.

We claim:

1. A method for screening for histone deacetylase enzyme activity in a test sample, comprising the steps of:
i) contacting said test sample with a liquid screening solution comprising a compound of Formula I: 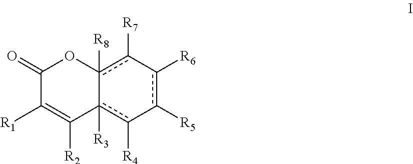
wherein:
R₁, R₂, R₃, R₄, R₈, R₆, R₇, and R₈ are independently selected from the group consisting of:
hydrogen,
hydroxyl,
carbonyl,
acetyl,
Ar,
straight or branched chain C₁-C₉ alkyl,
straight or branched chain C₁-C₉ alkyl substituted with one or more halo, trifluoromethyl, nitro, C₁-C₈ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,
straight or branched chain C₂-C₉ alkenyl or alkynyl, and
straight or branched chain C₂-C₉ alkenyl or alkynyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,
provided that at least one of R₁, R₂, R₃, R₄, R₅, R₆, R₇, and R₈ must be —O—C(O)—R₉, wherein R₉ is straight or branched chain C₁-C₃ alkyl; and
Ar is a mono-, bi- or tricyclic, carbo- or heterocyclic ring, wherein the ring is either unsubstituted or substituted in one or more position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, or amino; wherein the individual ring sizes are 5-6 members; and wherein the heterocyclic ring contains 1-6 heteroatom(s) selected from the group consisting of O, N, and S; and
ii) assaying for fluorescence produced by a product of histone deacetylase enzyme activity in said liquid screening solution.

2. The method of claim 1, wherein said Ar is selected from the group consisting of naphthyl, indolyl, thioindolyl, furyl, thiazolyl, thienyl, pyridyl, quinolinyl, isoquinolinyl, fluorenyl, phenyl, and benzyl.

3. The method of claim 2, wherein said Ar is phenyl.

4. The method of claim 1, wherein R₆ is —O—C(O)—R₉.

5. The method of claim 4, wherein R₆ is acetyl.

6. The method of claim 5, wherein R₁, R₂, R₃, R₄, R₅, R₇, and R₈ are independently selected from the group consisting of:
hydrogen,
hydroxyl,
carbonyl,
acetyl,
Ar,
straight or branched chain C₁-C₃ alkyl, and
straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

7. The method of claim 6, wherein R₁, R₂, R₃, R₄, R₅, R₇, and R₈ are independently selected from the group consisting of:
hydrogen,
hydroxyl,
carbonyl,
acetyl,
Ar,
straight or branched chain C₁-C₃ alkyl, and
straight or branched chain C₁-C₃ alkyl substituted with halo, trifluoromethyl, nitro, hydroxy, phenoxy, benzyloxy, amino, or Ar.

8. The method of claim 7, wherein R₁, R₂, R₃, R₄, R₅, R₇, and R₈ are independently selected from the group consisting of:
hydrogen,
methyl,
hydroxyl,
carbonyl,
acetyl, and
phenyl.

9. The method of claim 1, wherein R₁, R₂, R₃, R₄, R₅, R₆, R₇, and R₈ are independently selected from the group consisting of:
hydrogen,
hydroxyl,
carbonyl,
acetyl,
Ar,
straight or branched chain C₁-C₃ alkyl, and
straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

10. The method of claim 1, wherein said compound is of Formula II: 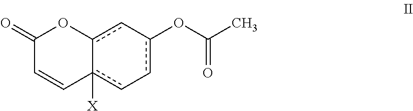
wherein X is selected from the group consisting of hydrogen, methyl, hydroxyl, carbonyl, acetyl, and phenyl.

11. The method of claim 10, wherein X is hydrogen or phenyl, and said assay for fluorescence is conducted at about 447 nm.

12. A method for screening for inhibitors of histone deacetylase enzyme activity, comprising the steps of:
i) contacting an inhibitor candidate compound or composition with a liquid screening solution comprising a histone deacetylase enzyme and a compound of Formula I: 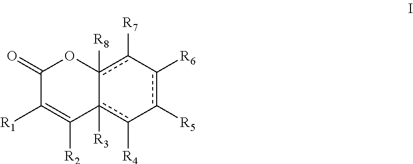
wherein:
R₁, R₂, R₃, R₄, R₅, R₆, R₇, and R₈ are independently selected from the group consisting of:
hydrogen,
hydroxyl,
carbonyl,
acetyl,
Ar,
straight or branched chain C₁-C₉ alkyl,
straight or branched chain C₁-C₉ alkyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,
straight or branched chain C₂-C₉ alkenyl or alkynyl, and
straight or branched chain C₂-C₉ alkenyl or alkynyl substituted with one or more halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar,
provided that at least one of R₁, R₂, R₃, R₄, R₅, R₆, R₇, and R₈ must be —O—C(O)—R₉, wherein R₉ is straight or branched chain C₁-C₃ alkyl; and
Ar is a mono-, bi- or tricyclic, carbo- or heterocyclic ring, wherein the ring is either unsubstituted or substituted in one or more position(s) with halo, hydroxyl, nitro, trifluoromethyl, C₁-C₆ straight or branched chain alkyl or alkenyl, C₁-C₄ alkoxy, C₁-C₄ alkenyloxy, phenoxy, benzyloxy, or amino; wherein the individual ring sizes are 5-6 members; and wherein the heterocyclic ring contains 1-6 heteroatom(s) selected from the group consisting of O, N, and S; and
ii) assaying for fluorescence produced by a product of histone deacetylase enzyme activity in said reaction solution.

13. The method of claim 12, wherein said Ar is selected from the group consisting of naphthyl, indolyl, thioindolyl, furyl, thiazolyl, thienyl, pyridyl, quinolinyl, isoquinolinyl, fluorenyl, phenyl, and benzyl.

14. The method of claim 13, wherein said Ar is phenyl.

15. The method of claim 12, wherein R₆ is —O—C(O)—R₉.

16. The method of claim 15, wherein R₆ is acetyl.

17. The method of claim 16, wherein R₁, R₂, R₃, R₄, R₅, R₇, and R₈ are independently selected from the group consisting of:
hydrogen,
hydroxyl,
carbonyl,
acetyl,
Ar,
straight or branched chain C₁-C₃ alkyl, and
straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

18. The method of claim 17, wherein R₁, R₂, R₃, R₄, R₅, R₇, and R₈ are independently selected from the group consisting of:
hydrogen,
hydroxyl,
carbonyl,
acetyl,
Ar,
straight or branched chain C₁-C₃ alkyl, and
straight or branched chain C₁-C₃ alkyl substituted with halo, trifluoromethyl, nitro, hydroxy, phenoxy, benzyloxy, amino, or Ar.

19. The method of claim 18, wherein R₁, R₂, R₃, R₄, R₅, R₇, and R₈ are independently selected from the group consisting of:
hydrogen,
methyl,
hydroxyl,
carbonyl,
acetyl, and
phenyl.

20. The method of claim 12, wherein R₁, R₂, R₃, R₄, R₅, R₆, R₇, and R₈ are independently selected from the group consisting of:
hydrogen,
hydroxyl,
carbonyl,
acetyl,
Ar,
straight or branched chain C₁-C₃ alkyl, and
straight or branched chain C₁-C₃ alkyl substituted with halo, trifluoromethyl, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, or Ar.

21. The method of claim 12, wherein said compound is of Formula II: 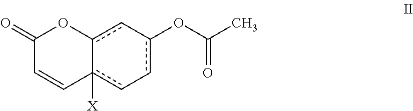
wherein X is selected from the group consisting of hydrogen, methyl, hydroxyl, carbonyl, acetyl, and phenyl.

22. The method of claim 21, wherein X is hydrogen or phenyl, and said assay for fluorescence is conducted at about 447 nm.


SEQUENCE LISTING (2 sequences)

SEQ ID NO: 1 - protein, 8 aa, Artificial Sequence
  Synthetic Sequence
GAKRHRKV

SEQ ID NO: 2 - protein, 36 aa, Artificial Sequence
  Synthetic Sequence
DAQTNSSSNNNNNNNNNNLIDTTENLYFQGSKHSDP